(19) United States
(12) Patent Application Publication
(10) Pub. No.: US 2007/0105756 A1
(43) Pub. Date: May 10, 2007

(54) Vancomycin formulations having reduced amount of histamine

(76) Inventors: Thomas B. May, Grayslake, IL (US); Richard Blessing, Vernon Hills, IL (US); Sukumaran K. Menon, Gurnee, IL (US); David H. Ostrow, Lake Zurich, IL (US)

Correspondence Address: BRIAN R. WOODWORTH, 275 N. FIELD DRIVE, DEPT. NLEG BLDG H-1, LAKE FOREST, IL, 60045-2579, US

(21) Appl. No.: 11/589,469

(22) Filed: Oct. 30, 2006

Related U.S. Application Data
(60) Provisional application No. 60/731,693, filed on Oct. 31, 2005.
(60) Provisional application No. 60/731,664, filed on Oct. 31, 2005.
(60) Provisional application No. 60/731,776, filed on Oct. 31, 2005.

(51) Int. Cl. A61K 38/14

(52) U.S. Cl. 514/8

(57) ABSTRACT

A pharmaceutical composition containing vancomycin that has been treated to remove histamine and a method of removing histamine from a vancomycin preparation containing histamine.

### CROSS REFERENCE TO RELATED APPLICATIONS

This application claims priority based upon U.S. Provisional Application Ser. No. 60/731,693 filed Oct. 31, 2005, U.S. Provisional Application Ser. No. 60/731,664 filed Oct. 31, 2005 and U.S. Provisional Application Ser. No. 60/731,776 filed Oct. 31, 2005, which are expressly incorporated herein by reference in their entirety.

### FIELD OF THE INVENTION

The invention relates to pharmaceutical compositions for treating bacterial infections. In particular, the invention is related to a vancomycin pharmaceutical composition having a reduced amount of histamine.

Description of Related Art

Vancomycin is a tricyclic glycopeptide antibiotic derived from Amycolatopsis orientalis (formerly Nocardia orientalis and Streptomyces orientalis). The glycopeptide has the chemical formula C₆₆H₇₅Cl₂N₉O₂₄.HCl. Vancomycin is used to treat infections by Gram positive bacteria. It is a primary treatment of infections by Methicillin Resistant Staphylococcus aureus (MRSA) or for Methicillin Sensitive S. aureus (MSSA) infections in β-lactam allergic patients. Vancomycin is an antibiotic of last resort. It is typically reserved for severe infections in order to prevent increased resistance to vancomycin in the population. Vancomycin is increasingly important owing to the emergence of bacteria with resistance to multiple anti-infectives.

Vancomycin dosing is typically three times daily. Dosing is usually by slow infusion in order to avoid two major side effects: phlebitis at the injection site and “Red Man Syndrome” (RMS). Phlebitis is typically resolved by suspending therapy and changing injection sites and/or changing from peripheral to PICC catheters. RMS is typically resolved by suspending therapy, administering an anti-histamine, and resuming therapy at slower infusion rates. RMS, also known as the “red-man”, “red man's”, “red neck” or “red person's syndrome”, is a commonly recognized adverse reaction of vancomycin administration. It is characterized by a complex of symptoms including: pruritis, urticaria, erythema, angioedema, tachycardia, hypotension, occasional muscle aches, and a maculopapular rash that usually appears on the face, neck, and upper torso. Cardiovascular toxicity may occur resulting in cardiac depression and cardiac arrest. Patients commonly begin to experience itching and warmth over their head and chest, with or without the development of a rash. The onset of RMS usually occurs within 30 minutes of the start of the infusion, but it may also occur after the infusion has ended. The reaction typically resolves between one and several hours after the end of the infusion. Hypotension, or low blood pressure, may also occur in the absence of other symptoms associated with RMS.

The precise cause of RMS is unknown. Despite the replacement of the old formulation of vancomycin, commonly described as “Mississippi Mud” due to its coloring attributable to impurities, newer and purer vancomycin products still produce side effects, including RMS. The rate of infusion recommended by the manufacturers of vancomycin is no greater than 10 mg/min, over at least one hour. Two hour infusions are typical owing to the potential for RMS to occur. The RMS reaction is usually associated with a rapid rate of infusion, but two cases of possible RMS have been reported after oral administration of vancomycin. Even at slower rates of infusion, vancomycin has caused hypotensive reactions.

Several studies have suggested that vancomycin directly causes histamine release as measured by increased plasma histamine level after vancomycin administration. This, however, would suggest that patients are demonstrating an allergy to vancomycin. A minority of patients may have true allergic reactions, as evidenced by reactions of greater intensity upon subsequent exposure to vancomycin. However, the etiology observed for the majority of patients suggests that RMS is not a true allergic reaction, i.e., RMS is not an IgE induced histamine release from mast cells. The reactions associated with RMS are not dependent on the duration of therapy; they may occur anytime during the infusion, and even occur for patients that have previously tolerated numerous doses of vancomycin. Patients can be re-administered vancomycin once the symptoms resolve, albeit at a slower rate. Therefore, the etiology of RMS is thought to be due to a non-immune related release of histamine, as histamine plasma concentrations have been shown to increase after the administration of vancomycin.

Dosing of vancomycin by infusion, and the concomitant need to actively monitor the side-effect profile, requires constant attention by the nursing staff. Therefore, the administration of vancomycin is complicated in an outpatient setting. A composition that allows for the bolus injection of vancomycin without causing phlebitis or Redman Syndrome is an unmet medical need.

### BRIEF DESCRIPTION OF THE FIGURES

FIG. 1 is a graph showing the separation of histamine from vancomycin using anion exchange chromatography. Vancomycin was monitored by absorbance at 280 nm. Fractions were collected and assayed for histamine using an ELISA.

FIG. 2 is a graph showing the separation of histamine from vancomycin using an anti-histamine affinity column. Vancomycin was monitored by absorbance at 280 nm. Fractions were collected and assayed for histamine using an ELISA.

FIG. 3 is a graph showing the separation of histamine from vancomycin using an anti-histamine affinity column. Vancomycin was monitored by absorbance at 280 nm. Fractions were collected and assayed histamine using an ELISA.

FIG. 4 shows the results of the determination of histamine in vancomycin samples using HPLC separation followed by mass spectrometry/mass spectrometry (MS/MS).

### DETAILED DESCRIPTION

As used herein, the singular forms “a,” “an”, and “the” include plural referents unless the context clearly dictates otherwise.

Vancomycin is a fermentation product of Amycolatopsis orientalis. It is possible that histamine or histamine-like compounds are present in the fermentation process. If so, a process that reduces the levels of these compounds, and the use of appropriate control limits for these compounds, could reduce or eliminate vancomycin side-effects. Pharmaceutical formulations of vancomycin with a reduced amount of histamine offer the advantages of a bolus injection with fewer side-effects, reduced nursing care, less morbidity and mortality, easier use in an outpatient setting, and the possibility of higher and/or faster dosing.

Vancomycin is produced by cultivating the bacteria A. orientalis in a nutrient culture media. The histamine or histamine-like compounds may be related to components present in the fermentation broth. Also, it is possible that intermediates of the vancomycin pathway or degradants of vancomycin are histamine-like. Histamine, phenylethylamine, tyramine, tryptamine, dopamine, and serotonin (5-hydroxytryptamine) are vasodialators or vasoactive compounds. Each of these compounds are derivatives of hydrophobic amino acids, ring structures with one or two rings, and planar in nature. Given that vancomycin is a glycopeptide built from hydrophobic amino acids, these vasoactive compounds may be intermediates or by-products of the synthetic pathway. Metal-induced or enzymatic catalysis could produce these compounds from vancomycin and represent vasoactive degradation products. The structure of vancomycin supports this hypothesis.

In a well characterized production process for vancomycin, the vancomycin fermentation broth is filtered and added to a column containing an adsorbent resin that decolorizes and desalts the vancomycin. The resin is washed, and the vancomycin is eluted with a solvent of low pH, followed by decolorization with carbon. The vancomycin eluant is then further purified using a crystallization step at low pH. The crystallized vancomycin is combined with a strong acid such as hydrochloric acid (HCl), and then precipitated in an organic solvent such as acetone to form vancomycin·HCl. This process for the manufacture and purification of vancomycin·HCl is disclosed in U.S. Pat. No. 3,067,099 to McCormick et al., which is incorporated its entirety by reference herein.

Typically, the desired vancomycin B is separated from vancomycin-related compounds and other impurities by elution of vancomycin broth through the absorbent column. Various resins are known to be selective for Vancomycin B. For example, DOWEX 50 WX2, a cation-exchange resin available from Dow Chemical, and AMBERLITE XAD-16, a non-functional resin available from Rohm & Haas, and others, have been utilized to separate vancomycin B from vancomycin-related compounds and impurities.

During the production of vancomycin, eluant from the columns is collected in fractions. Each fraction is analyzed to determine the concentration and quantity of vancomycin B. In this way, the fractions with the greatest concentration of vancomycin B can be combined to optimize the yield from the process. The purity of the vancomycin varies from fraction to fraction and depends on a number of factors such as the solvent used to elute the vancomycin from the column and the fermentation medium. In one method of the production of vancomycin described in U.S. Pat. No. 5,258,495, which is incorporated herein by reference in its entirety, the selected vancomycin eluate(s) is combined with an ammonium chloride solution to obtain a solution having a pH of about 2.0 to about 3.5. The solution is then crystallized before being redissolved in a basic solution. An acid is again added to the vancomycin before a final crystallization step.

Vancomycin for parenteral administration is provided in a lyophilized form, which is reconstituted at the time of administration with sterile water. The lyophilized product is reconstituted with 20 mL of water for every gram of vancomycin and then subsequently diluted in sterile saline or dextrose solutions for infusion. Dosage for vancomycin for parenteral administration is generally 2 grams per day divided as either 500 mg every 6 hours, or 1 gram every 12 hours. To avoid side effects, such as RMS, phlebitis and hypotension, infusion rates of no more than 10 mg per minute for adult patients with normal renal function are recommended. Each dose is administered over the course of at least sixty minutes. Two hour infusions are more typical.

Vancomycin from manufacturers representing over 50% of the worldwide market and over 80% of the US market were analyzed for the presence of histamine. Lot testing included results from various bulk drug vendors as well as finished dosage forms. As shown in Table 1, each of these products contained over 40 nM histamine in the reconstituted formula.

TABLE 1

| | | Histamine |
|---|---|---|
| Vanco Lot | Vendor | (nM) |
| 953963A | A | 43.35 |
| 041109 | B | 56.43 |
| 041110 | B | 63.08 |
| 041111 | B | 67.47 |
| 041205 | B | 63.65 |
| 041206 | B | 65.62 |
| 041207 | B | 73.42 |
| WM15082 | C | 67.27 |
| 933203A | A | 55.69 |
| A3230596 | D | 53.57 |
| A3230605 | D | 47.58 |
| A3230607 | D | 54.26 |
| 040706 | E | 56.16 |
| 040602 | E | 53.29 |
| 040511 | E | 51.30 |
| 895003A | A | 47.00 |
| 895003A | A | 58.80 |
| 895003A | A | 54.47 |

In each of these analyses, commercial samples of vancomycin were reconstituted according to the label at 50 mg/mL. Bulk drug samples were reconstituted in sterile water at 50 mg/mL. samples were tested using the Histamine EIA Kit from SPIBio (Massy Cedex, France) according to the directions of the manufacturer.

The histamine concentration found in each of the samples is known to be biologically active by the oral route in sensitive individuals (i.e., >5 nM). Activity would be expected to be greater by the injection route, where the histamine is theoretically 100% bioavailable, and likely sufficiently active to cause a histamine response in normal individuals. None of the manufacturers of these commercial samples have previously reported that the histamine is present in vancomycin. The realization that histamine is present in these samples allows for the preparation of a formulation that does cause many of the side effects that may be caused by the histamine present in the formulations.

Ordinary analytical procedures used during the vancomycin purification process have not detected histamine for several reasons. For instance, histamine does not have a strong chromophore and is not observed by UV spectroscopy typically used to monitor the vancomycin purification process. Also, vancomycin presents a complex chromatographic profile due to numerous related compounds. Any histamine peak in the profile may be masked or associated with a different impurity. Indeed, histamine concentrations are extremely low from the perspective of chemical detection. Therefore, the presence of histamine in vancomycin products could easily be overlooked at the levels present in vancomycin.

In one aspect, the invention is directed to a pharmaceutical composition including vancomycin that is substantially free of histamine. Substantially free means that the amount of histamine in the composition does not produce the unwanted, histamine-related side effects associated with the administration of vancomycin, including phlebitis, RMS, and low blood pressure. In various aspects of the invention, the pharmaceutical composition includes vancomycin and less than 40 nM histamine, or alternative less than 30 nM, 20 nM, or 10 nM histamine, in vancomycin when reconstituted from a lyophilized powder to provide a solution of one gram of vancomycin per 20 mL of solution. In one aspect, the invention is directed to a vancomycin formulation having less than about 0.90 ng histamine per mg of vancomycin. For example, no more than about than 0.80, 0.70, 0.60, 0.50, 0.40, 0.30, 0.20 and 0.10 ng histamine per mg of vancomycin.

In another aspect, the invention is directed to a pharmaceutical composition of a vancomycin that has been treated to remove histamine. Histamine can be removed from vancomycin by any number of ways known to those of skill in the art of pharmaceutical purification, including gel filtration, ion exchange (cation or anion) exchange chromatography, affinity chromatography, immunoaffinity chromatography, and crystallization processes. While one or more of these methods, and usually cation exchange chromatography and crystallization, is presently used for purification of commercial preparations of vancomycin, the process has not been controlled to remove histamine to a level that it is not physiologically significant in patients receiving vancomycin.

Histamine can be removed from vancomycin by loading a vancomycin product on an anion exchange column, and eluting the histamine separate from vancomycin. In particular, a column that is a strong anion exchanger used with a linear gradient of a basic buffer and an acid buffer. For example, 0.25 M ammonium hydroxide and 1 N acetic acid will separate histamine from vancomycin on a strong anion exchange column; the vancomycin will bind to the column under basic conditions while the histamine can be eluted. Acid conditions will elute the vancomycin to provide a clear separation of the two compounds. In the alternative, conditions can be adjusted so that the histamine binds to the column and the vancomycin is eluted first. Lower strength anion exchange and cation exchange columns may also be suitable but may be less efficient depending upon the histamine load and the separation capabilities.

Immunoaffinity chromatography is also suitable for removing histamine from vancomycin. Anti-histamine antibody (IgG) when coupled to a suitable column will bind the histamine and not the vancomycin. After vancomycin is washed from the column, histamine can be eluted with a suitable solvent.

In addition, under the appropriate conditions, gel filtration, amino-affinity columns, and crystallization are all techniques that can be used to separate histamine from vancomycin. Gel filtration conditions should account for the relatively small size of vancomycin.

In another aspect, the invention is directed to a method for treating a patient suffering from a condition treatable with vancomycin. The method includes administering to the patient an effective amount of the pharmaceutical composition of vancomycin that has a reduced amount of histamine. While vancomycin is typically reserved as an antibiotic of last resort to prevent the development of vancomycin resistant bacterial strains, vancomycin is an effective antibiotic against a variety of infections, as is well documented in the literature. Most commonly, vancomycin is used to treat infections caused by Methicillin Resistant Staphylococcus aureus (MRSA) or Methicillan Sensitive S. aureus (MSSA). Use of vancomycin is increasingly important due to the emergence of bacterial strains with multiple antibiotic resistances. The ability to bolus inject vancomycin will substantially reduce the patient burden for the nursing staff.

In another aspect, the invention is directed to a method for reducing the histamine related side-effects associated with administration of vancomycin. These side effects are well documented, and include phlebitis at an infusion site, blood pressure drop, and RMS. The administration of a vancomycin having a reduced amount of histamine can reduce or prevent these side effects.

In another aspect, the invention is related to a growth media having a reduced amount of histamine relative to conventional media, and that supports growth of Amycolatopsis orientalis and the fermentation of vancomycin. Regardless of the media, manufacturing specifications can be provided to ensure vancomycin formulations are produced and tested to ensure that the formulation has less than 40 nM histamine.

The following are provided for exemplification purposes only and are not intended to limit the scope of the invention described in broad terms above. All references cited in this disclosure are incorporated herein by reference.

### EXAMPLES

### Example 1

### Chromatographic Separation of Histamine from Vancomycin

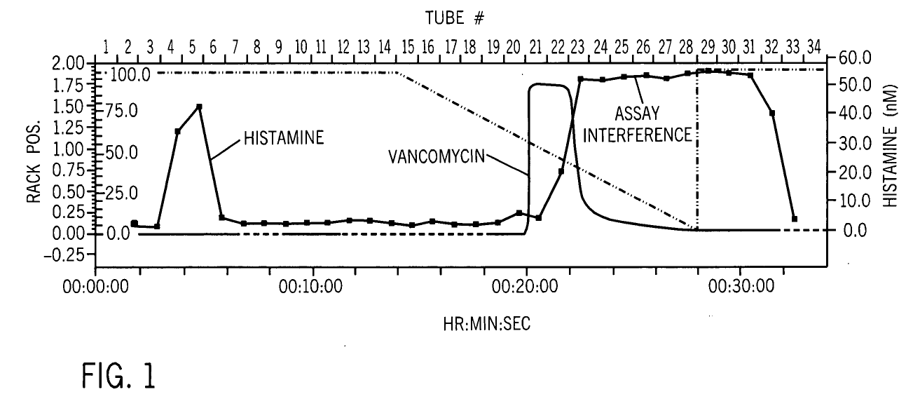

Vancomycin (Hospira, Inc.) was reconstituted at 50 mg/mL per the label directions and then adjusted to 0.25 M ammonium hydroxide using a 1 M stock solution. A 5 mL HighQ column (BioRad), a strong anion exchanger, was installed on the Biologic DuoFlow chromatography system and equilibrated with 0.25 M ammonium hydroxide mobile phase. Vancomycin was loaded onto the column via a 1 mL injection loop and the column was washed with a 30 mL isocratic step at a flow rate of 2.5 mL/minute. Vancomycin was then eluted with a 35 mL linear gradient of 0.25 M ammonium hydroxide to 1 N acetic acid. UV (A_{280 nm} ), pH and conductivity were monitored during the chromatography. Column fractions (2.5 mL) were assayed for histamine by ELISA (SPI-Bio) and vancomycin by UV. FIG. 1 confirmed the presence of histamine in vancomycin samples and demonstrated that chromatographic separation of vancomycin (fractions 21-22) and histamine (fractions 4-5) was possible. The acetic acid interfered with histamine ELISA's starting at fraction 21. Fractions 21 and 22 demonstrated less than 10 nM histamine when pH was adjusted above pH 7.

### Example 2

### Anti-Histamine Affinity Column Chromatography

Anti-histamine rabbit antibody (Sigma) was coupled to Affi-Gel Hz resin (BioRad) per the kit instructions. A 2 mL column contained approximately 0.47 mg of anti-histamine antibody. The column was equilibrated with five volumes of 10 mM HEPES (pH 7.0) buffer.

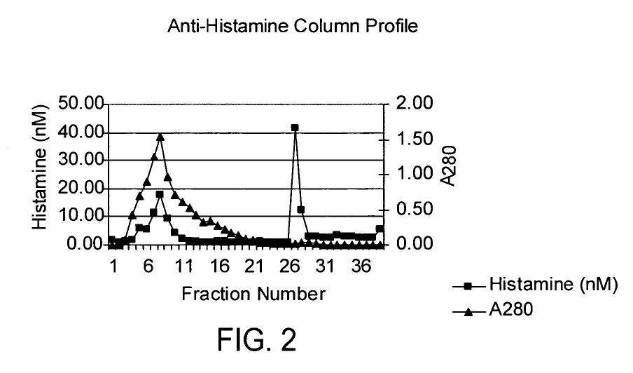

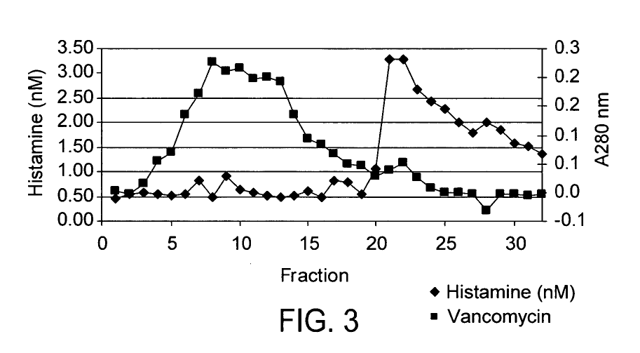

Vancomycin was reconstituted at 50 mg/mL in HEPES buffer and a 2 mL aliquot was loaded onto the column. The column was washed with one volume of HEPES buffer containing 0.5 M sodium chloride followed with two volumes of HEPES buffer. The bound histamine was then eluted with 2 volumes of 0.1 N acetic acid. Fractions (0.5 mL) were collected and then assayed by UV and ELISA. FIG. 2 shows the presence of antibody bound histamine. The residual histamine in the vancomycin peak likely resulted from overloading of the column. The vancomycin peak fractions were combined and run a second time on the anti-histamine affinity column. As depicted in FIG. 3, these results showed that the vancomycin peak lacked histamine and that the additional histamine present in the initial material was separated from the vancomycin.

### Example 3

### Determination of histamine by mass spectroscopy

Fractions representing the histamine peak from multiple runs of the HighQ column as in Example 1 were collected, lyophilized, and reconstituted in small volume of water. Chromatographic separation was accomplished using gradient high-performance liquid chromatography. The liquid chromatograph (Thermo Finnigan Surveyor) was operated at 1.0 mL/min with the following gradient profile:

| Time | % Mobile | % Mobile |
|---|---|---|
| (minutes) | Phase A | Phase B |
| 0 | 100 | 0 |
| 12.0 | 0 | 100 |
| 16.0 | 0 | 100 |
| 16.8 | 100 | 0 |
| 24.0 | 100 | 0 |

Mobile Phase A was prepared by mixing 50 mL of HPLC grade water (Burdick and Jackson) with 950 mL of acetonitrile (EMD). Mobile Phase B was prepared by mixing 670 mL of 12.5 mM ammonium acetate (EM Science), 0.72 mL of glacial acetic acid (EMD) and 330 mL of acetonitrile. Both mobile phases were degassed using an inline vacuum degasser. The chromatography column was a Hypersil APS-2, 150×3 mm with 3-micron particle size. The column temperature was maintained at 60 degrees Celsius. The injection volume was 100 microliters. Authentic samples of histamine were prepared by dissolving histamine dihydrochloride (Fluka) in HPLC grade water, and diluting.

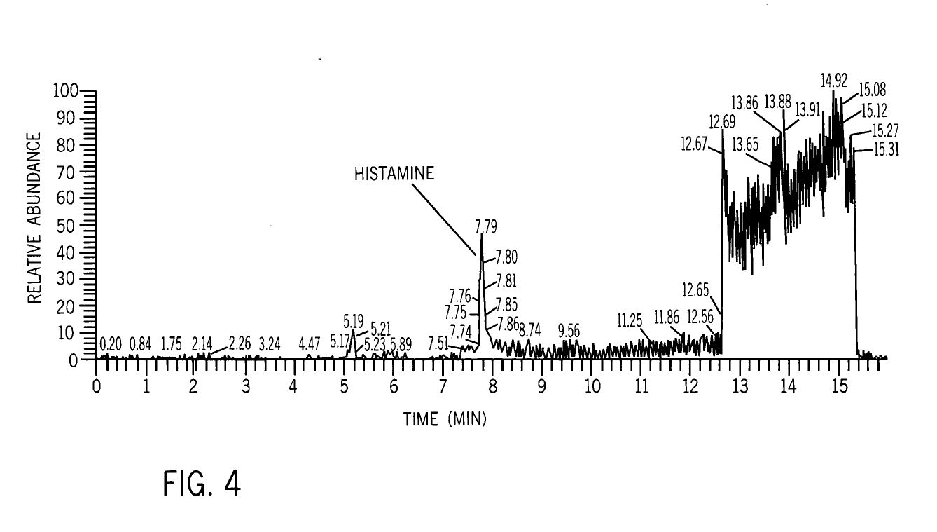

A triple quadrupole mass spectrometer (Thermo Finnigan Quantum Ultra) was used in single reaction monitoring mode (SRM) to monitor the transition from m/z 112 to m/z 95 (loss of neutral ammonia from protonated histamine) with positive ion electrospray ionization. As shown in FIG. 4, a peak was observed after about 7.79 minutes having a mass of 95.2. This peak was consistent with the retention time and mass observed with histamine dihydrochloride standards run under the same conditions (data not shown). Thus, the data confirmed the ELISA results indicating that histamine is present in vancomycin. Histamine was concentrated 5- to 10-fold for chemical detection as compared to the concentrations necessary for detection by ELISA.

Although various specific embodiments of the present invention have been described herein, it is to be understood that the invention is not limited to those precise embodiments and that various changes or modifications can be affected therein by one skilled in the art without departing from the scope and spirit of the invention.

What is claimed is:

1. A pharmaceutical composition comprising vancomycin and less than 40 nM histamine.

2. The pharmaceutical composition of claim 1 comprising less than 30 nM histamine

3. The pharmaceutical composition of claim 1 comprising less than 20 nM histamine

4. The pharmaceutical composition of claim 1 comprising less than 10 nM histamine.

5. A histamine free pharmaceutical composition of vancomycin.

6. A pharmaceutical composition comprising a vancomycin that has been treated to remove histamine.

7. The pharmaceutical composition of claim 6 wherein the composition comprises less than 40 nM histamine.

8. The pharmaceutical composition of claim 7 wherein the composition comprises less than 30 nM histamine.

9. The pharmaceutical composition of claim 8 wherein the composition comprises less than 20 nM histamine.

10. The pharmaceutical composition of claim 9 wherein the composition comprises less than 10 nM histamine.

11. The pharmaceutical composition of claims 1-10 comprising no more than 0.90 nanogram histamine per milligram of vancomycin.

12. A method of treating a patient suffering from a condition treatable with vancomycin comprising administering to the patient an effective amount of the pharmaceutical composition of any of claims 1-11.

13. The method of claim 12 wherein the condition is an infection caused by Methicillan Resistant Staphylococcus aureus (MRSA) or Methicillan Sensitive S. aureus (MSSA).

14. A method for reducing side-effects associated with administration of vancomycin comprising administering to a patient in need of therapy a pharmaceutical composition of any of claims 1-11.

15. The method of claim 14 wherein the side-effects are phlebitis at an infusion site, blood pressure drop, and red man syndrome.

16. The method of claim 14 wherein the vancomycin is treated by anion exchange chromatography or affinity chromatography to separate the histamine from the vancomycin.

17. A method of removing histamine from a vancomycin preparation containing histamine, the method comprising using chromatography to separate histamine from the vancomycin preparation.

18. The method of claim 17 wherein the chromatography comprises anion exchange chromatography.

19. The method of claim 18 wherein the chromatography comprises a strong anion exchange column.

20. The method of claim 17 wherein the chromatography comprises affinity chromatography.

21. The method of claim 20 wherein the affinity chromatography comprises capturing histamine on a column comprising an anti-histamine antibody.

22. A histamine-free growth media that supports growth of Amycolatopsis orientalis and the fermentation of vancomycin.

23. A pharmaceutical composition comprising vancomycin that provides a reduced incidence of RMS, phlebitis and hypotension upon administration to a patient.

24. A pharmaceutical composition comprising vancomycin for bolus injection that provides a reduced incidence of RMS, phlebitis and hypotension upon administration to a patient.

25. A pharmaceutical composition comprising vancomycin that is produced in under specifications requiring histamine of less then 40 nM.

26. A pharmaceutical composition comprising vancomycin that is produced in under specifications requiring less than 0.9 ng histamine per milligram of vancomycin.

27. A manufacturing process for reducing histamine in vancomycin pharmaceutical formulations comprising fermenting Amycolatopsis orientalis in a medium having a reduced amount of histamine.

28. A manufacturing process for reducing histamine in vancomycin pharmaceutical formulations comprising purifying vancomycin to remove histamine.
